(19) United States
(12) Patent Application Publication
(10) Pub. No.: US 2018/0221643 A1
(43) Pub. Date: Aug. 9, 2018

(54) INFUSION LINE CLAMP SYSTEMS FOR INFUSION PUMPS

(71) Applicant: Smiths Medical ASD, Inc., Plymouth, MN (US)

(72) Inventors: Clinton Robert HETCHLER, Minnetonka, MN (US); Steve COTE, Stillwater, MN (US)

(73) Assignee: Smiths Medical ASD, Inc., Plymouth, MN (US)

(21) Appl. No.: 15/749,459

(22) PCT Filed: Jul. 8, 2016
(86) PCT No.: PCT/US2016/041523

§ 371 Date: Jan. 31, 2018

Related U.S. Application Data
(60) Provisional application No. 62/199,718, filed on Jul. 31, 2015.

(51) Int. Cl. A61M 39/28; A61M 5/145; A61M 5/168

(52) U.S. Cl. CPC A61M 39/281; A61M 2005/16863; A61M 5/16877; A61M 5/1456

(57) ABSTRACT

An infusion line clamp system includes a clamp configured to attach to an infusion line and reversibly occlude the infusion line. The clamp is biased to occlude the infusion line except when acted upon by an externally applied opening force. The system also includes an infusion pump having a control system, a clamp receptacle configured to releasably secure the clamp to the infusion pump, and a clamp actuation mechanism that is configured to selectively actuate the clamp under command of the control system, by selective application of opening force to the clamp when the clamp is secured to the pump.

### CROSS-REFERENCE TO RELATED APPLICATIONS

The present application is a National Phase entry of PCT Application No. PCT/US2016/041523, filed on Jul. 8, 2016, which claims priority to US Provisional Patent Application No. 62/199,718, filed on Jul. 31, 2015, which are hereby fully incorporated herein by reference.

### TECHNICAL FIELD

This disclosure relates generally to infusion pumps, and more particularly, to infusion line clamp systems for infusion pumps.

### BACKGROUND

Infusion pumps are useful medical devices for managing the delivery and dispensation of many types of therapeutic infusates. Infusion pumps provide significant advantages over manual administration by accurately delivering infusates over an extended period of time. Infusion pumps are particularly useful for treating diseases and disorders that require regular pharmacological intervention, including cancer, diabetes, and vascular, neurological, and metabolic disorders. They also enhance the ability of healthcare providers to deliver anesthesia and manage pain. Infusion pumps are used in various settings, including hospitals, nursing homes, and other short-term and long-term medical facilities, as well as in residential care settings. There are many types of infusion pumps, including ambulatory, large volume, patient-controlled analgesia (PCA), elastomeric, syringe, enteral, and insulin pumps. Infusion pumps can be used to administer medication through various delivery methods, including intravenously, intraperitoneally, intra-arterially, intradermally, subcutaneously, in close proximity to nerves, and into an intraoperative site, epidural space or subarachnoid space.

Often, when deploying an infusion pump for initial use with a particular patient or during various other procedures such as, for example, replacing a syringe in the pump, a hemostat, slide clamp, or other separate mechanical clamping device can be employed to clamp or otherwise pinch and occlude the infusion line in a portion where the infusate exits the pump and leads to the patient. This separate task is typically performed so that, for example, infusate does not leak out of infusion line not yet connected to a patient or cause an unintended bolus delivery of infusate to a patient who is connected to the line. Such manual tasks add to caregiver workloads and can cause difficulties in infusion protocols due to, for example, time needed to find hemostats or other clamping devices, manipulate them for placement on and occlusion of proper portions of infusion line, and then remove them after the intended occlusions are no longer needed. Furthermore, inconvenience or even hazards can be introduced if the hemostat or other separate mechanical clamping device is inadvertently not removed after starting the infusion pump, resulting at a minimum in an alarm that may be triggered and causing disruption and inefficiency in the patient's treatment or other procedure involving the pump as well. Therefore it would be advantageous to provide integrated clamping and occlusion functionality with infusion pumps, to obviate a need for hemostats or other separate mechanical clamping devices and thereby minimize disruptions and inefficiencies in infusion protocols.

### SUMMARY

This disclosure relates to infusion pumps, and more particularly, to systems and methods for securing infusion lines to infusion pumps.

In an embodiment, an infusion line clamp system includes a clamp configured to attach to an infusion line and reversibly occlude the infusion line. The clamp is biased to occlude the infusion line except when acted upon by an externally applied opening force. The system also includes an infusion pump having a control system, a clamp receptacle configured to releasably secure the clamp to the infusion pump, and a clamp actuation mechanism that is configured to selectively actuate the clamp under command of the control system, by selective application of opening force to the clamp when the clamp is secured to the pump.

In an embodiment, an infusion line clamp system includes a clamp configured to attach to an infusion line and reversibly occlude the infusion line. The clamp is biased to occlude the infusion line except when acted upon by an externally applied opening force. The system also includes an infusion pump having a control system, a clamp receptacle configured to releasably secure the clamp to the infusion pump, and a clamp actuation mechanism that is configured to selectively actuate the clamp under command of the control system, by selective application of opening force to the clamp when the clamp is secured to the pump. The infusion line clamp system is configured such that when the clamp is removed from the clamp receptacle, any opening force that had been applied by the clamp actuation mechanism to the clamp is removed from the clamp and the clamp then responsively occludes, or remains occluding, the infusion line.

In an embodiment, an infusion line clamp system includes a clamp configured to attach to an infusion line and reversibly occlude the infusion line. The clamp is biased to occlude the infusion line except when acted upon by an externally applied opening force. The system also includes an infusion pump having a control system, a clamp receptacle configured to releasably secure the clamp to the infusion pump, and a clamp actuation mechanism that is configured to selectively actuate the clamp under command of the control system, by selective application of opening force to the clamp when the clamp is secured to the pump. The clamp actuation mechanism includes a solenoid or a motor.

In an embodiment, an infusion line clamp system includes a clamp configured to attach to an infusion line and reversibly occlude the infusion line. The clamp is biased to occlude the infusion line except when acted upon by an externally applied opening force. The system also includes an infusion pump having a control system, a clamp receptacle configured to releasably secure the clamp to the infusion pump, and a clamp actuation mechanism that is configured to selectively actuate the clamp under command of the control system, by selective application of opening force to the clamp when the clamp is secured to the pump. The infusion pump is a syringe pump.

In an embodiment, an infusion line clamp system includes a clamp configured to attach to an infusion line and reversibly occlude the infusion line. The clamp is biased to occlude the infusion line except when acted upon by an externally applied opening force. The system also includes an infusion pump having a control system, a clamp receptacle configured to releasably secure the clamp to the infusion pump, and a clamp actuation mechanism that is configured to selectively actuate the clamp under command of the control system, by selective application of opening force to the clamp when the clamp is secured to the pump. The control system is configured and programmed, upon occurrence of a predetermined safety condition, to remove opening force from the clamp via the clamp actuation mechanism, thereby occluding the infusion line.

In an embodiment, an infusion line clamp system includes a clamp configured to attach to an infusion line and reversibly occlude the infusion line. The clamp is biased to occlude the infusion line except when acted upon by an externally applied opening force. The system also includes an infusion pump having a control system, a clamp receptacle configured to releasably secure the clamp to the infusion pump, and a clamp actuation mechanism that is configured to selectively actuate the clamp under command of the control system, by selective application of opening force to the clamp when the clamp is secured to the pump. The control system is configured and programmed, upon occurrence of a predetermined safety condition, to remove opening force from the clamp via the clamp actuation mechanism, thereby occluding the infusion line. The infusion pump includes an accelerometer that is communicatively coupled to the control system; and the predetermined safety condition is detected at least in part based upon acceleration information measured by the accelerometer.

In an embodiment, an infusion pump includes a control system and a clamp receptacle that is configured to releasably secure a clamp to the pump. The clamp is configured to attach to an infusion line and reversibly occlude the infusion line; and the clamp is biased to occlude the infusion line except when acted upon by an externally applied opening force. A clamp actuation mechanism is configured to selectively actuate the clamp under command of the control system by selective application of opening force, when the clamp is secured to the pump. A syringe receptacle is configured to receive a syringe; and the syringe is coupleable to the infusion line. The infusion pump also includes a pusher mechanism that can act to move a plunger of the syringe.

In an embodiment, an infusion pump includes a control system and a clamp receptacle that is configured to releasably secure a clamp to the pump. The clamp is configured to attach to an infusion line and reversibly occlude the infusion line; and the clamp is biased to occlude the infusion line except when acted upon by an externally applied opening force. A clamp actuation mechanism is configured to selectively actuate the clamp under command of the control system by selective application of opening force, when the clamp is secured to the pump. A syringe receptacle is configured to receive a syringe; and the syringe is coupleable to the infusion line. The infusion pump also includes a pusher mechanism that can act to move a plunger of the syringe. The clamp actuation mechanism includes a solenoid or a motor.

In an embodiment, an infusion pump includes a control system and a clamp receptacle that is configured to releasably secure a clamp to the pump. The clamp is configured to attach to an infusion line and reversibly occlude the infusion line; and the clamp is biased to occlude the infusion line except when acted upon by an externally applied opening force. A clamp actuation mechanism is configured to selectively actuate the clamp under command of the control system by selective application of opening force, when the clamp is secured to the pump. A syringe receptacle is configured to receive a syringe; and the syringe is coupleable to the infusion line. The infusion pump also includes a pusher mechanism that can act to move a plunger of the syringe. The control system is configured and programmed, upon occurrence of a predetermined safety condition, to remove opening force from the clamp via the clamp actuation mechanism, thereby occluding the infusion line.

In an embodiment, an infusion pump includes a control system and a clamp receptacle that is configured to releasably secure a clamp to the pump. The clamp is configured to attach to an infusion line and reversibly occlude the infusion line; and the clamp is biased to occlude the infusion line except when acted upon by an externally applied opening force. A clamp actuation mechanism is configured to selectively actuate the clamp under command of the control system by selective application of opening force, when the clamp is secured to the pump. A syringe receptacle is configured to receive a syringe; and the syringe is coupleable to the infusion line. The infusion pump also includes a pusher mechanism that can act to move a plunger of the syringe. The infusion pump also includes an accelerometer that is communicatively coupled to the control system; and the predetermined safety condition is detected at least in part based upon acceleration information measured by the accelerometer.

In an embodiment, a method of administering an infusate includes attaching a clamp to a clamp receptacle of an infusion pump. The clamp is attached to, and reversibly occludes, a portion of an infusion line prior to attachment to the pump. The portion of the infusion line is configured to deliver infusate to or from the infusion pump. The method also includes reversing occlusion of the portion of the infusion line by the clamp, including application of an opening force onto the clamp by a clamp actuation mechanism of the infusion pump. The clamp actuation mechanism applies the opening force in response to a control system of the infusion pump.

In an embodiment, a method of administering an infusate includes attaching a clamp to a clamp receptacle of an infusion pump. The clamp is attached to, and reversibly occludes, a portion of an infusion line prior to attachment to the pump. The portion of the infusion line is configured to deliver infusate to or from the infusion pump. The method also includes reversing occlusion of the portion of the infusion line by the clamp, including application of an opening force onto the clamp by a clamp actuation mechanism of the infusion pump. The clamp actuation mechanism applies the opening force in response to a control system of the infusion pump. The act of reversing occlusion includes a user providing input to the control system of the infusion pump, that results in the control system commanding the clamp actuation mechanism to apply opening force to the clamp.

In an embodiment, a method of administering an infusate includes attaching a clamp to a clamp receptacle of an infusion pump. The clamp is attached to, and reversibly occludes, a portion of an infusion line prior to attachment to the pump. The portion of the infusion line is configured to deliver infusate to or from the infusion pump. The method also includes reversing occlusion of the portion of the infusion line by the clamp, including application of an opening force onto the clamp by a clamp actuation mechanism of the infusion pump. The clamp actuation mechanism applies the opening force in response to a control system of the infusion pump. The act of reversing occlusion includes the control system commanding the clamp actuation mechanism to apply opening force to the clamp, when pre-determined clamp opening conditions are met.

In an embodiment, a method of administering an infusate includes attaching a clamp to a clamp receptacle of an infusion pump. The clamp is attached to, and reversibly occludes, a portion of an infusion line prior to attachment to the pump. The portion of the infusion line is configured to deliver infusate to or from the infusion pump. The method also includes reversing occlusion of the portion of the infusion line by the clamp, including application of an opening force onto the clamp by a clamp actuation mechanism of the infusion pump. The clamp actuation mechanism applies the opening force in response to a control system of the infusion pump. The method also includes, subsequent to reversing occlusion of the portion of the infusion line by the clamp, re-occluding the portion of infusion line by the clamp, including removal or reduction of opening force.

In an embodiment, a method of administering an infusate includes attaching a clamp to a clamp receptacle of an infusion pump. The clamp is attached to, and reversibly occludes, a portion of an infusion line prior to attachment to the pump. The portion of the infusion line is configured to deliver infusate to or from the infusion pump. The method also includes reversing occlusion of the portion of the infusion line by the clamp, including application of an opening force onto the clamp by a clamp actuation mechanism of the infusion pump. The clamp actuation mechanism applies the opening force in response to a control system of the infusion pump. The method also includes, subsequent to reversing occlusion of the portion of the infusion line by the clamp, re-occluding the portion of infusion line by the clamp, including removal or reduction of opening force. Removal or reduction of opening force is commanded by the control system.

In an embodiment, a method of administering an infusate includes attaching a clamp to a clamp receptacle of an infusion pump. The clamp is attached to, and reversibly occludes, a portion of an infusion line prior to attachment to the pump. The portion of the infusion line is configured to deliver infusate to or from the infusion pump. The method also includes reversing occlusion of the portion of the infusion line by the clamp, including application of an opening force onto the clamp by a clamp actuation mechanism of the infusion pump. The clamp actuation mechanism applies the opening force in response to a control system of the infusion pump. The method also includes, subsequent to reversing occlusion of the portion of the infusion line by the clamp, re-occluding the portion of infusion line by the clamp, including removal or reduction of opening force. The control system commands removal or reduction of opening force upon occurrence of a predetermined safety condition.

In an embodiment, an infusion line clamp includes a first member and a second member separated by a space into which a portion of a compressible infusion line can be placed. A biasing mechanism is configured to urge the first and second members together such that the infusion line is compressively clamped between the first and second members with sufficient force to temporarily and reversibly occlude the infusion line. Mounting structures are configured to interface with a clamp receptacle of an infusion pump, such that the infusion line clamp can be releasably secured to the infusion pump. At least one opening force receiving portion is configured to receive opening force exerted by a clamp actuation mechanism of the infusion pump. When sufficient opening force is exerted on the at least one opening force receiving portion, the biasing mechanism is overcome and the first and second members are moved apart to open the infusion line to fluid flow.

In an embodiment, operation of an infusion pump includes a pump-actuatable clamp system for an infusion pump and a method of operation selected from a group as disclosed and described herein of: preventing “crosstalk”; improving startup performance; running a pump motor in reverse to pull backwardly on a syringe plunger and thereby mitigate any unintended bolus of infusate that would otherwise be delivered from the syringe; providing a test of the pump motor; providing a test of a downstream occlusion sensor; providing a test of motor health; providing a test of motor rate error prevention; determining a presence and amount of air in infusion line; determining whether there may be a leak or a misconnection somewhere in the infusate's flow path; estimating an internal diameter of a syringe; and estimating a fluid volume capacity of a syringe.

The above summary is not intended to describe each and every example or every implementation of the disclosure. The Description that follows more particularly exemplifies various illustrative embodiments.

### BRIEF DESCRIPTION OF THE DRAWINGS

The following description should be read with reference to the drawings. The drawings, which are not necessarily to scale, depict several examples and are not intended to limit the scope of the disclosure. The disclosure may be more completely understood in consideration of the following description with respect to various examples in connection with the accompanying drawings, in which:

FIG. 1 is a schematic front perspective view of an infusion pump;

FIG. 2 is a schematic perspective view of a pump-actuatable clamp in an open, non-occluding configuration;

FIG. 3 is a schematic perspective view of the pump-actuatable clamp of FIG. 2 in a closed, occluding configuration;

FIG. 4 is a schematic detail perspective view of a portion of an infusion system that includes an infusion pump having a clamp receptacle, and the pump-actuatable clamp of FIGS. 2 and 3;

FIG. 5 is a flow diagram of a method of administering an infusate;

FIG. 6 is a schematic perspective view of the pump-actuatable clamp of FIGS. 2, 3, and 4 from another perspective; and

FIG. 7 is a schematic perspective view of the pump-actuatable clamp of FIGS. 2, 3, 4, and 6 from another perspective.

### DETAILED DESCRIPTION

The present disclosure is related to U.S. Patent Application Publication No. 2013/0123579, “ILLUMINATED TUBING SET,” U.S. Patent Application Publication No. 2013/0123743, “MEDICAL TUBING DETECTION AND MANAGEMENT,” U.S. Provisional Patent Application Ser. No. 62/086,551, “SYRINGE INFUSION PUMP SECURITY,” U.S. Provisional Patent Application Ser. No. 62/153,203, “SYSTEMS AND METHODS FOR PROVIDING NOTE-TAKING SURFACES ON INFUSION PUMPS,” WIPO Application No. PCT/US2015/013049, “Pump Startup Algorithms and Related Systems and Methods,” U.S. Provisional Patent Application Ser. No. 62/169,115, “INFUSATE TUBING CLAMP SYSTEMS FOR INFUSION PUMPS,” and U.S. Provisional Patent Application Attorney Docket No. P4436US00, “SYSTEMS AND METHODS FOR SECURING AN INFUSION LINE,” all of which are hereby incorporated by reference in their entireties.

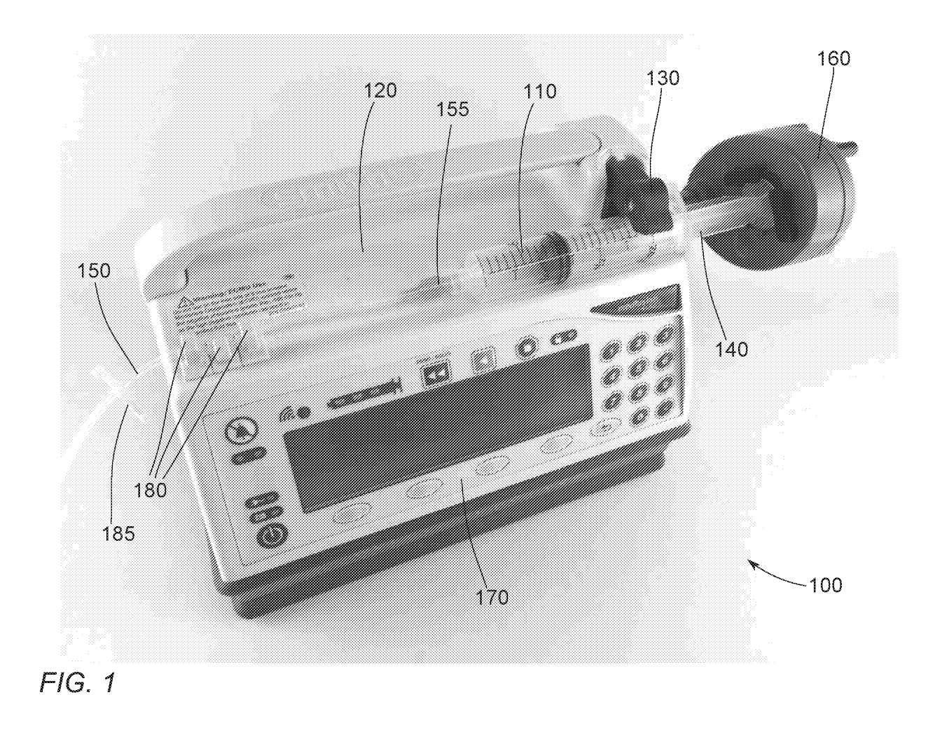

FIG. 1 illustrates an infusion pump 100, such as, for example, a MEDFUSION 4000 infusion pump from Smiths Medical ASD, Inc. In general, infusion pump 100 can be a syringe-type pump that can be used to deliver various infusates, drug therapies and treatments to patients. When in use, infusion pump 100 typically can include a removable and replaceable pharmaceutical container or syringe 110, which can be supported on and secured to housing 120 of pump 100 and can be secured thereto by clamp 130. In some embodiments, syringe 110 can be separately supplied from pump 100. In other embodiments, syringe 110 can be an integrated component of pump 100. Syringe 110 can include a plunger 140 that can force fluid outwardly from syringe 110 via infusion line 150 that is connected to a patient (not illustrated). Infusion line 150 can be fluidically and mechanically coupled to syringe 150 at an attachment point 155, typically at the tip of the syringe opposite the plunger 140, from which infusate is dispensed. A pusher or plunger driver mechanism 160, when in operation, can act to move plunger 140 of syringe 110. Operation of mechanism 160 can be provided by way of, for example, cooperative action of a motor and lead screw arrangement internal to housing 120 of pump 100. In some embodiments, a sensor can be provided (not shown; typically internal to plunger driver mechanism 160) that can monitor force and/or plunger position in the syringe according to system specifications.

Pump 100 typically can include a user interface 170 (that can include display screen, keypad, and any other suitable user interface components) for relaying commands to a control system (not illustrated) of pump 100. User interface 170 generally can allow a user to enter various parameters, including but not limited to names, drug information, limits, delivery shapes, information relating to hospital facilities, as well as various user-specific parameters (e.g., patient age and/or weight). Infusion pump 100 can include any appropriate wired or wireless input/output (I/O) interface port and/or protocol (including, but not limited to, USB, Ethernet, WiFi, NFC, Bluetooth, and the like) for connecting pump 100 to a network or computer (not illustrated) having software designed to interface with pump 100. Power to infusion pump 100 can be provided via an AC or DC power cord, from an internally provided battery source (not illustrated), or by any other suitable means. Embodiments can also include a wireless power source (not illustrated). User inputs to infusion pump 100 can be provided by programming from a user, such as a patient, pharmacist, scientist, drug program designer, medical engineer, nurse, physician, or other medical practitioner or healthcare provider. User inputs may utilize direct interfacing (via, e.g., keyboards, touch screens, or other touch-based inputs) as shown, and/or user inputs may utilize indirect or “touchless” interfacing (i.e., gestures; voice commands; facial movements or expressions; finger, hand, head, body and arm movements; or other inputs that do not require physical contact such as cameras, sensors of electric field, capacitance, or sound). User inputs generally can be interfaced, communicated, sensed, and/or received by operator input mechanisms of user interface 170.

Pump 100 can include tube or line holders 180, for guiding and removably securing a portion of infusion line 150 to housing 120 of pump 100. In some embodiments, and as illustrated, line holders 180 can include multiple hooks facing opposite directions into which infusion line 150 can be threaded, such threading generally being performed manually by a practitioner. Line holders 180 can provide an important safety function for pump 100. In usage, undesirable tension can be introduced (often unintentionally) in infusion line 150. In such situations, line holders 180 can help maintain the proper securement of syringe 110 to pump 100. As discussed in greater detail in co-pending U.S. Provisional Patent Application Attorney Docket No. P4436US00, “SYSTEMS AND METHODS FOR SECURING AN INFUSION LINE,” line holders 180 can, in effect, relieve or redirect tension applied to the infusion line such that such tension is unlikely or unable to disturb the securement of the syringe 110 to pump 100. Also illustrated in FIG. 1, a manually-manipulable clamp 185 can be used with infusion line 150 to reversibly occlude the infusion line. Manually-manipulable clamp 185 can take a variety of forms, such as a slide clamp, as illustrated, or a hemostat, etc. Manually-manipulable clamp 185 can be used to intentionally and reversibly occlude infusion line 150, for example, when replacing a syringe 110 in pump 100, or in any other suitable situation when it may be desired or necessary to occlude the infusion line.

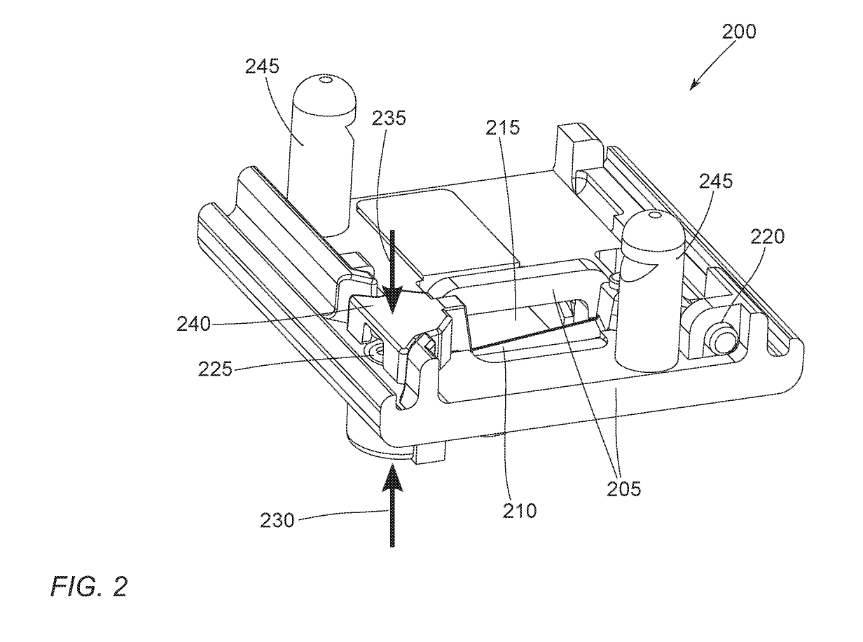

As clamp 185 can require manual manipulation by a user such as a practitioner, it can provide a workload burden and can cause potentially erroneous operation or hazard. The clamp systems and methods of the present disclosure seek to obviate these potential disadvantages of manual clamps for infusion systems, as well as provide other advantages. FIG. 2 is a schematic perspective view of a pump-actuatable clamp 200 of the present disclosure. Clamp 200 is merely exemplary, and those of ordinary skill in the art will readily appreciate that other implementations of clamp mechanisms could be employed in the systems and methods of the present disclosure.

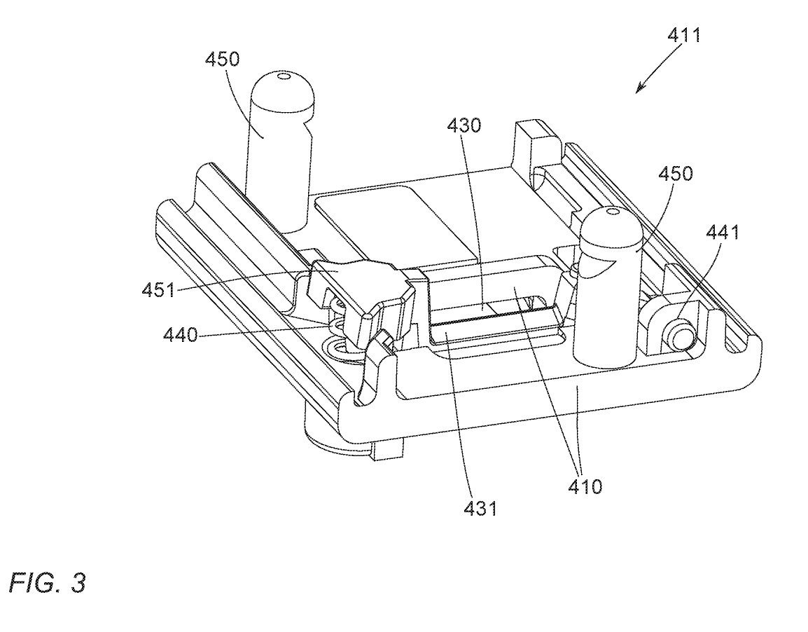

Pump-actuatable clamp 200 can include a first member 205 and a second member 210 separated by a space 215 through and within which a portion of a compressible infusion line (not shown) can be placed. Pump-actuatable clamp 200 can be structured and configured such that first and second members 205, 210 are movable relative to each other such that the dimensions of space 215 therebetween can vary depending on the relative positions of the members. First and second members 205, 210 of clamp 200 as shown can be mechanically coupled by a hinge 220, but this is not limiting and other mechanical arrangements are possible for making first and second members movable relative to each other. As illustrated in FIG. 2, first and second members 205, 210 can be positioned such that an infusion line extending through space 215 can be substantially not compressed, thus permitting fluid flow through the infusion line. With reference to FIGS. 2 and 3, clamp 200 can include a biasing mechanism 225, such as a spring, configured to urge the first and second members 205, 210 together such that the infusion line is compressively clamped between the first and second members with sufficient force to temporarily and reversibly occlude the infusion line. FIG. 3 is, in particular, a schematic perspective view of clamp 200 in which the biasing mechanism 225 has urged the first and second members 205, 210 together, narrowing space 215 sufficient to occlude an infusion line. While the space or separation 215 between first and second members 205, 210 as shown in FIG. 3 has a substantially non-zero value, this illustration merely shows the relative positions that the first and second members might take when biased together sufficiently to occlude a particular size and type of infusion line (for clarity, the infusion line is not shown in FIG. 2 or 3). The illustrated relative positions of first and second members 205, 210 in FIG. 3 do not necessarily represent the closest possible relative position of the two members (i.e., smallest possible space 215 between the members) to which the biasing mechanism 225 can urge the members. Pump-actuatable clamp 200 can be constructed such that space 215 can be arbitrarily small, with biasing mechanism 225 being capable of urging first and second members 205, 210 together with sufficient force, so that a wide range of infusion line sizes and compressibilities can be occluded by the clamp. It is believed that clamp systems and methods of the present disclosure can be practiced with any compressible infusion line.

With reference again to FIG. 2, first and second members 205, 210 of pump-actuatable clamp 200 are illustrated as being spaced apart such that space 215 may be sufficiently sized to allow placement of an infusion line therein without compressively occluding the line. In order for the members to be so positioned, an opening force or set of forces can be applied to the clamp to overcome the force exerted by the biasing mechanism 225 that urges the first and second members 205, 210 together. An example set of opening forces are indicated schematically in FIG. 2 by illustrative force arrows 230, 235. Note that force arrows 230 and 235 are shown as being directed in generally opposing directions, with arrow 230 positioned to indicate a force exerted on first member 205 and arrow 235 positioned to indicate a force exerted on second member 210. Opening force can be exerted on second member 210 at an opening force-receiving portion 240, which can be structured to be actuatable manually (for example, by being pressed with a finger) and/or by a mechanical or electro-mechanical device or machine, as described further elsewhere herein.

Pump-actuatable clamp 200 can be structured such that opening forces can be exerted manually on the clamp as aforementioned, to allow manual opening and closing of the clamp. In the present disclosure, it is further contemplated that clamp 200 can be mechanically secured, mounted, or otherwise attached to an infusion pump, and that the pump can selectively apply opening force(s) to the pump-actuatable clamp sufficient to selectively actuate the clamp. Pump-actuatable clamp 200 can include mounting structures 245 configured to mate to corresponding structures of an infusion pump. While mounting structures 245 are depicted as notched posts in FIG. 2, any suitable mounting structures, components, scheme, or technique can be employed.

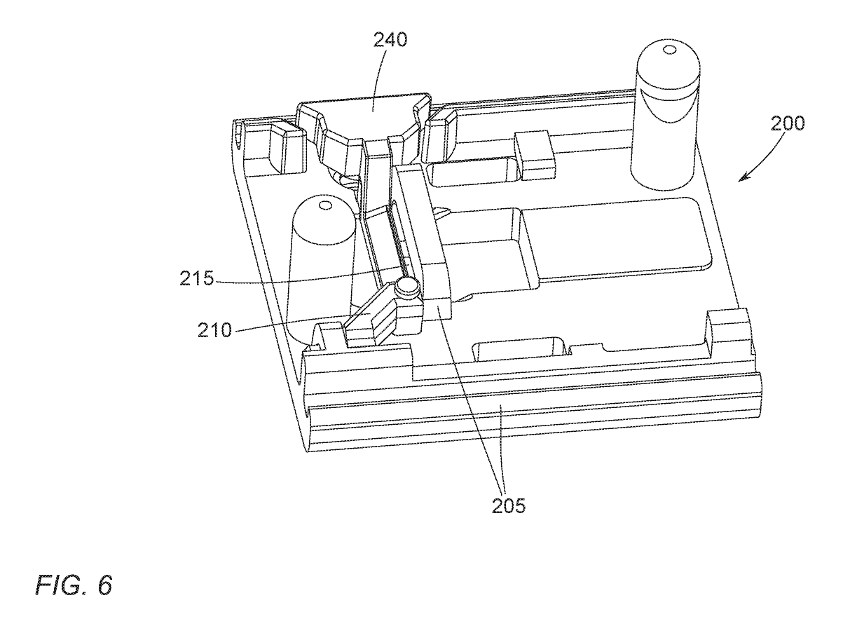

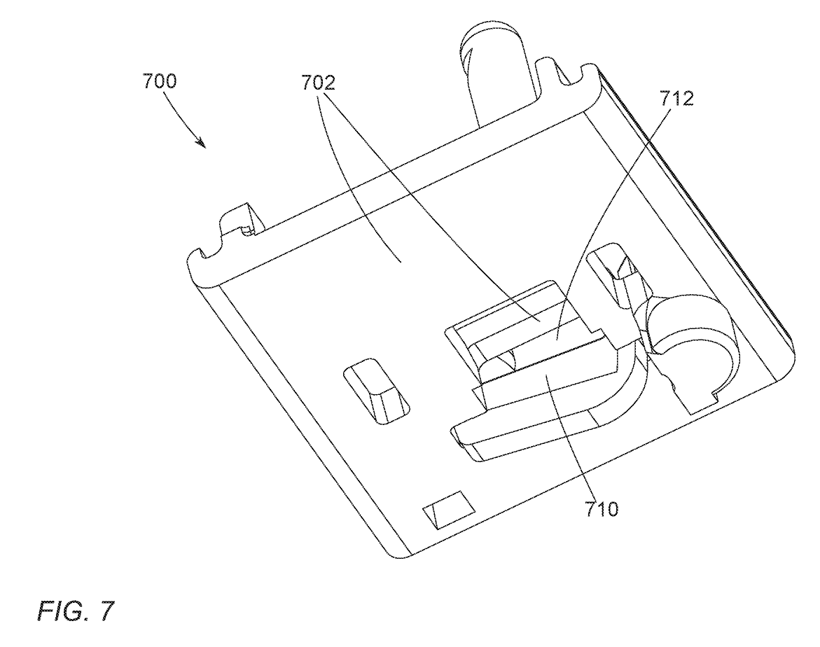

Referring now to FIGS. 6 and 7, schematic perspective views of clamp 200 are illustrated from different viewpoints to afford further understanding of the structure and arrangement of this particular implementation of a pump-actuatable clamp. In FIG. 6, clamp 200 is shown with first and second members 205, 210 in the relatively more closed configuration of FIG. 3. In FIG. 7, clamp 200 is shown with first and second members 205, 210 in the relatively more open configuration of FIG. 2, and is rendered generally from the opposite side of FIGS. 2, 3, and 6.

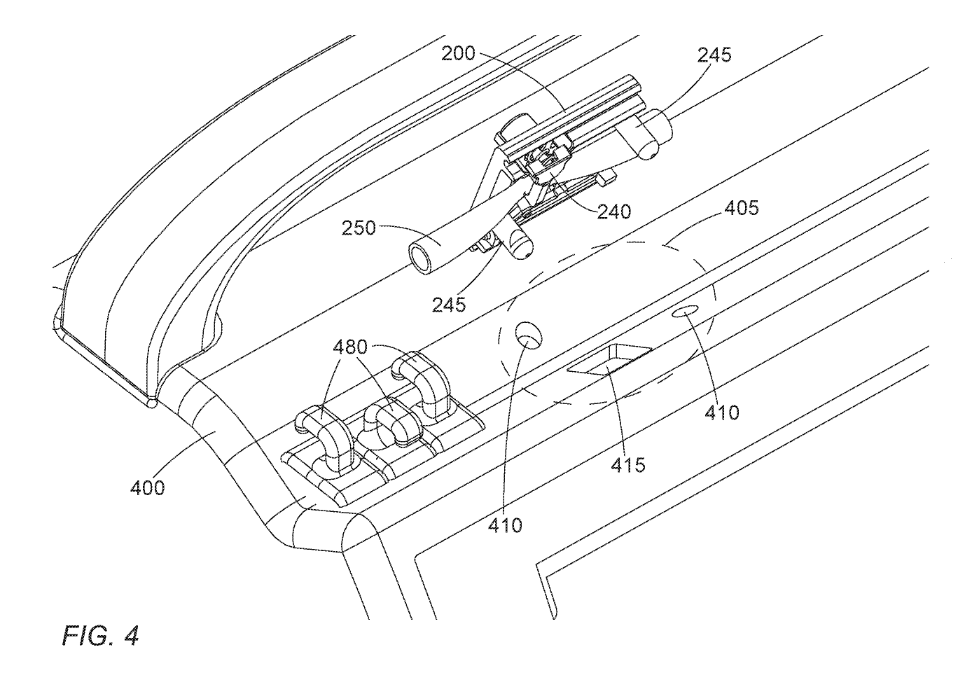

Referring now to FIG. 4, a schematic detail perspective view illustrates a portion of an infusion system that includes an infusion pump 400 that can include a clamp receptacle 405 configured to releasably secure clamp 200 to the pump. Pump-actuatable clamp 200 is shown attached to a portion 250 of an infusion line (such as, for example, line 150 of FIG. 1), which passes through space 215, such that clamp 200 can reversibly occlude the line. The extent of portion 250 of the infusion line is limited in the drawing only for ease and clarity of illustration. Although not shown explicitly in FIG. 4, it is to be understood that the infusion line can be fluidically coupled to a syringe or other infusate reservoir from which infusion pump 400 can be configured to pump infusate via the infusion line. Pump-actuatable clamp 200 is shown in FIG. 4 in a vicinity of clamp receptacle 405, but not attached to clamp receptacle 405. By comparison of corresponding features, discussed further herein, it will be apparent to persons having ordinary skill in the art how clamp 200 can be secured to clamp receptacle 405.

Infusion pump 400 can be any suitable infusion pump, and can be an infusion pump similar to infusion pump 100 of FIG. 1, but with the addition of clamp receptacle 405, a feature that is absent from the illustration of pump 100 in FIG. 1. Infusion pump 400 can be a syringe-type pump, although the systems and methods of the present disclosure are not limited to syringe pumps and can be practiced with any suitable infusion pump.

Clamp receptacle 405 of infusion pump 400 can include attachment structures 410 (in the illustrated embodiment, sockets) corresponding to, and configured to mate to, mounting structures 245 (posts) of pump-actuatable clamp 200. Clamp receptacle 405 can be configured to releasably secure clamp 200 to pump 400, via mounting structures 245 of clamp 200 and attachment structures 410 of clamp receptacle 405, or via any other suitable securement arrangement. Mounting structures 245 of clamp 200 and attachment structures 410 of clamp receptacle 405 can be structured and configured to releasably attach or mate in any suitable manner. As an example, clamp receptacle 405 could include a locking mechanism such as latches or catches (not illustrated) within sockets of attachment structures 410 configured to reversibly lock the posts of mounting structures 245. In some embodiments, clamp receptacle 405 can include a sensing mechanism communicatively coupled to the control system of the pump, to detect/verify that clamp 200 is secured to receptacle 405. Although the example of clamp 200 illustrated in FIGS. 2, 3, and 4 is depicted as including two mounting structures 245, it is to be appreciated and understood that any suitable numbers of mounting structures may be provided in a particular embodiment of an infusion line clamp system. It is to be further appreciated and understood that, in a particular embodiment of an infusion line clamp system, one or more of mounting structures 245 could be “keyed” with, for example, a longitudinal rib that would slidingly engage a slot or slots in one or more corresponding sockets of attachment structures 410 respectively.

Pump 400 can include line holders 480 through which an infusion line (such as line 150 of FIG. 1) can be threaded for relief or redirection of tension in the line. However, when clamp 200 is attached to a portion 250 of infusion line and is secured to clamp receptacle 405, tension in the line can be relieved or redirected by the attached clamp, potentially making redundant the act of threading the line through line holders 480. Eliminating the step of manually threading the line through line holders 480 can beneficially reduce the workload for a practitioner. Pump 400 may include line holders 480 nonetheless for use when an infusion set is used without a clamp like clamp 200 that is securable to the pump at the clamp receptacle 405. In some embodiments, an infusion line could be attached to a non-clamp device that is, however, securable to pump 400 at clamp receptacle 405, for stress relief and/or other functions.

Infusion pump 400 can include a clamp actuation mechanism configured to selectively actuate clamp 200, under command of the control system of the pump, by selective application of opening force. The clamp actuation mechanism can be capable of selectively actuating clamp 200 when the clamp is secured to the pump via clamp receptacle 405. As illustrated in FIG. 4, the clamp actuation mechanism can include an opening 415 into the housing of pump 400 into which opening force-receiving portion 240 of second member 210 of clamp 200 can extend. The clamp actuation mechanism can include a movable force-applying device (not shown) located in or operably proximate to opening 415 in pump 400 that can be driven to extend and retract so as to apply or not apply opening force to opening force-receiving portion 240 of second member 210 of pump-actuatable clamp 200, when the clamp is secured to the pump in clamp receptacle 405. The movable force-applying device can be driven by any suitable mechanism, such as (but not limited to) an electromechanical motor or solenoid (not illustrated), under the command of the control system of the pump.

In some embodiments, when no clamp is attached to clamp receptacle 405, the movable force-applying device can be retracted, such that when a pump-actuatable clamp 200 is subsequently mated to the pump at the receptacle, the movable force-applying device does not apply opening force to opening force-receiving portion 240 of second member 210 of clamp 200 as the clamp is secured to the pump. In some such embodiments, only later, under the control and command of the control system of the pump, would the movable force-applying device be extended so as to apply opening force to opening force-receiving portion 240 of pump-actuatable clamp 200. In other embodiments, when no clamp is attached to clamp receptacle 405, the movable force-applying device can be extended, such that when a pump-actuatable clamp 200 is subsequently mated to the pump at the receptacle, the movable force-applying device does apply opening force to opening force-receiving portion 240 of clamp 200 as the clamp is secured to the pump, thereby reversing occlusion of the portion 250 of infusion line. In still other embodiments, a pump can be contemplated that incorporates a clamp receptacle that does not have a clamp actuation mechanism. In some such embodiments, the clamp receptacle can be structured such that when a clamp like clamp 200 is secured to the pump, opening force is always applied by the clamp receptacle to open the clamp and reverse occlusion of the infusion line.

While FIGS. 2, 3, and 4 illustrate aspects of a particular embodiment of a pump-actuatable clamp and infusion pump system, it is to be appreciated and understood by those of ordinary skill in the relevant arts that other physical implementations of systems of infusion pumps and pump-actuatable clamps are possible within the scope of the present disclosure. Different clamping methods to occlude a tube can be used, and different ways of attaching a pump-actuatable clamp to a pump, and of actuating the clamp under the control system of the pump, can be envisioned in accordance with subject matter hereof The systems and methods of the present disclosure are contemplated for use with syringe pumps, but are not limited for use with such pumps. In some embodiments, an infusion pump operable with a pump-actuatable clamp can be a so-called large-volume pump that can deliver infusate from, for example, an intra-venous (IV) bag that can store a relatively large volume of infusate (in relation to a volume storable by, for example, a syringe).

It is also to be appreciated and understood that, as described by example or otherwise contemplated herein, pump-actuatable clamps can be provided separately from an infusion line, or can be provided together with the infusion line as part of an infusion set. In some cases, it is contemplated that a clamp could be opened to allow loading of an infusion line, and/or in some cases, an infusion line could be guided or “threaded” through a clamp, both actions being performable, potentially, by an end user such as a medical practitioner. In some cases, when provided with an infusion line as part of an infusion set, a pump-actuatable clamp can be non-detachable from the infusion set. In some cases, it may be desirable to provide a non-detachable clamp with each infusion set to provide consistent clamping or reversible occlusion performance over the lifetime of the single infusion set for which the clamp is intended for use.

Pump-actuatable clamp 200, as illustrated in FIGS. 2, 3 and 4, can be configured such that its default state, as biased by biasing mechanism 225 (without externally-applied actuation or opening force), is clamped, occluded, or otherwise closed. This may be desirable in some uses to prevent free-flow, or other unintended flow, of infusate in an infusion line before an infusion system is configured to provide controlled delivery of infusate to a patient as aforedescribed. In some other embodiments, a pump-actuatable clamp with a default, unactuated state of being unclamped, un-occluded, or otherwise opened, can be provided.

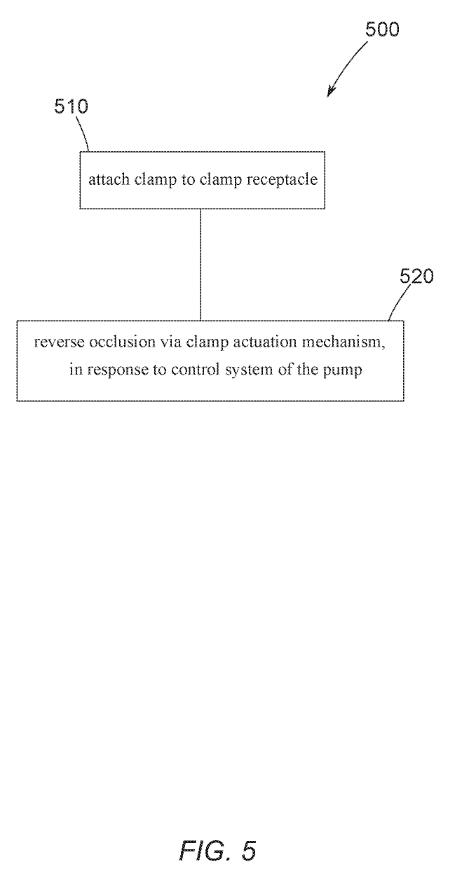

FIG. 5 is a flow diagram of a method 500 of administering an infusate. Method 500, while described in relation to pump-actuatable clamp 200 and infusion pump 400, can be practiced with any compatible combination of pump-actuatable clamp and infusion pump. Method 500 can include at 510 attaching pump-actuatable clamp 200 to clamp receptacle 405 of infusion pump 400. Although not illustrated in FIG. 5, clamp 200 can be attached to a portion 250 of infusion line configured to deliver infusate to or from the infusion pump 400 prior to attachment to the pump as aforedescribed. Although not illustrated in FIG. 5, prior to attachment to pump 400, clamp 200 can be biased by biasing mechanism 225 to be in a state where it is occluding the portion of infusion line 250.

At 520, method 500 can include reversing occlusion of the portion 250 of infusion line by the clamp 200. This can include application of an opening force onto the clamp 200 by a clamp actuation mechanism of the infusion pump 400 (more specifically, it can include application of opening force to opening force-receiving portion 240 of the clamp). The application of opening force by the clamp actuation mechanism can be in response to a command from the control system of the infusion pump 400. Although not illustrated in FIG. 5, subsequent to reversing occlusion at 520, pump 400 could, if appropriate, commence infusate delivery through the non-occluded infusion line.

In various embodiments, the control system of infusion pump 400 can issue commands to apply, or cease to apply, opening force by the clamp actuation mechanism for any suitable reason. In some embodiments, a user can provide input to the control system of infusion pump 400 that results in the control system commanding the clamp actuation mechanism to apply opening force to clamp 200. In some embodiments, the control system of pump 400 can command the clamp actuation mechanism to apply opening force to clamp 200 when pre-determined clamp opening conditions are met. The pre-determined clamp opening conditions can be any suitable conditions. The conditions can include, for example, systems of the pump 400 satisfactorily passing startup or other tests and/or routines. More extensive descriptions of pump operations that include clamp actuation and/or release are provided elsewhere herein.

In various embodiments, although not illustrated in FIG. 5, at a time after reversing occlusion at 520, the portion 250 of infusion line can be re-occluded by the clamp 200. In some such cases, removal or reduction of the opening force applied to the clamp 200 can result in the biasing mechanism 225 biasing the clamp back to a line-occluding state. Removal or reduction of the opening force applied to the clamp 200, and thus re-occlusion of the infusion line, can occur when the clamp is released and moved away from the clamp receptacle 405. Such a release and removal of the clamp 200 may occur as a routine matter, after completion of an infusion and subsequent rearrangement of infusion system components. It also could occur by accident, in which case the re-occlusion of the infusion line could perform a safety function.

Removal or reduction of the opening force applied to the clamp 200, and thus re-occlusion of the infusion line, also can occur when the clamp actuation mechanism removes or reduces opening force applied to the clamp, in response to a command or commands of the control system of the pump. Such a command or commands could be issued by the control system upon occurrence of a predetermined safety condition. In some embodiments, pump 400 can include an accelerometer (not shown) communicatively coupled to the control system. The predetermined safety condition that leads to issuance of a command to remove opening force from clamp 200 can be detected at least in part based upon acceleration information measured by the accelerometer. A sensed acceleration could be indicative of an unintended physical occurrence at pump 400 such as a sudden movement or impact which could potentially cause a disconnection of the infusion line from the pump, a leak from infusion line not yet connected to a patient, or an unintended bolus delivery of infusate to a patient who is connected to the infusion line. Other scenarios are contemplated in which acceleration measurement may indicate situations in which unintended physical movement, impact, or other unintentional force at the pump could occur, and in response to which re-occlusion of the infusion line may be commanded, such as (but not limited to) during patient transport in land vehicles across rough terrain or in aircraft through turbulent air. In some cases, the accelerometer can include its own controller, processor, or control system, etc., that can transmit a command or actuation signal to the clamp actuation system independent of a master control system of the pump. For the purposes of this disclosure, “control system” in relation to an infusion pump should be interpreted to include any such systems and/or sub-systems that can operate to control pump systems.

Although not specifically illustrated herein, it is also to be appreciated and understood that pump-actuatable clamp systems for infusion pumps—as described by example or otherwise contemplated herein—could be useful for prevention of so-called “crosstalk” between separate infusates being delivered to a patient. For example, when two drugs are highly incompatible but are both being delivered to a patient in a “piggybacking” infusion protocol, there may be a risk of migration of one drug to the other. To mitigate this risk a pump-actuatable clamp system could be activated manually, or automatically by the system, upon potential occurrence of such crosstalk by way of a suitable sensor and control technique (not illustrated).

Also although not specifically illustrated herein, it is also to be appreciated and understood that pump-actuatable clamp systems for infusion pumps—as described by example or otherwise contemplated herein—could be useful for improving startup performance. In such an embodiment, a pump-actuatable clamp system could continue occluding the infusion line with the syringe plunger driven by the pump until a known pressure is achieved. The pump-actuatable clamp could then be released therefore starting the infusion with no or minimal delay. For example, in an embodiment of improved startup performance provided by a pump-actuatable clamp system for an infusion pump, the pump would use its occlusion pressure sensor to detect a first pressure with the clamp closed/occluding the tubing and the pump drive idle. The pump's motor would then be run to advance the syringe plunger until the pump's occlusion pressure sensor detects a second pressure that is of a selected higher pressure than the first pressure. A suitable microprocessor would be employed by or in the pump, to calculate the second pressure for a particular use of improved startup performance provided by the pump-actuatable clamp system. In an embodiment, such calculation could take into account selected physical parameters such as certain syringe and/or tubing characteristics and thereby infer that the syringe plunger has been advanced far enough to effectively remove mechanical “slack”, “play”, or “backlash” from the drive train of the pump. In an embodiment, it could be possible to simply infer with reference to a pressure sensor coupled to the syringe or tubing that such slack, play, or backlash has been satisfactorily accommodated. As such, irrespective of a particular embodiment, force placed on the syringe plunger could be advantageously increased in a significantly shorter amount of time than if the motor simply ran (i) at its intended rate, as described in WIPO Application No. PCT/US2015/013049, filed on 27 Jan., 2015, and titled “Pump Startup Algorithms and Related Systems and Methods” (with the disclosure of this PCT application being incorporated herein, by reference thereto) or perhaps (ii) at a nominal rate for a predetermined time interval without benefit of pressure feedback information. After a selected time following occurrence of sensing the second pressure, the motor would then be stopped and the pump-actuatable clamp would be opened, to remove the occlusion of the tubing. In an embodiment, the selected time and the actions of stopping the motor and opening the clamp would be controlled by the aforementioned microprocessor. It is also to be appreciated and understood that in an embodiment of a pump-actuatable clamp system for an infusion pump, the system could command the pump's motor to run in reverse to pull backwardly on the plunger and thereby mitigate, reduce, or eliminate any unintended bolus of infusate that would otherwise be delivered to the patient due to, for example, a pressure that exceeded an optimal pressure for improved startup performance.

Also although not specifically illustrated herein, it is to be appreciated and understood that pump-actuatable clamp systems for infusion pumps—as described by example or otherwise contemplated herein—could provide several other internal features in addition to the aforedescribed user-facing features. For example, clamping the infusion line and increasing pressure within the tubing by activating the pump's motor to drive the pump could be used as a self-test of both the pump's motor and a downstream occlusion sensor. Increasing the pressure even further could be used as a test of motor health or motor rate error prevention. Furthermore, a suitable embodiment of a pump-actuatable clamp system could be used to determine a presence and amount of air in the infusion line. In this regard, the amount of air present in the infusion line could be roughly calculated by clamping the tubing and measuring how far the syringe has to travel before a specified pressure is reached. For example, in an embodiment of air detection provided by a pump-actuatable clamp system for an infusion pump, the system could function analogously to the aforedescribed improved startup performance feature. In such an embodiment of air detection, the pump would use its occlusion pressure sensor to detect a first pressure. With the pump-actuatable clamp occluding the tubing, the pump's motor would then be run to advance the pump's syringe plunger driver until the occlusion pressure sensor detects a second pressure that is of a selected higher pressure than the first pressure. A suitable microprocessor would be employed by or in the pump, to calculate the second pressure versus forward displacement of the pump's syringe plunger driver for a particular use of air detection provided by the infusion line clamp system. In an embodiment, such calculation could take into account selected physical parameters such as certain syringe and/or tubing characteristics. A forward displacement of the syringe plunger driver that does not result in an increase of pressure to the second pressure that was expected or predicted by the microprocessor would thereby result in a conclusion that air may be present in the tubing, and/or there may be a leak or a misconnection somewhere in the infusate's flow path.

Also although not specifically illustrated herein, it is also to be appreciated and understood that pump-actuatable clamp systems for infusion pumps—as described by example or otherwise contemplated herein—could provide yet another feature. For example, clamping the infusion line and increasing pressure within the syringe by activating the pump could be used to estimate the syringe's internal diameter and fluid volume capacity. Such estimate of the internal diameter could be used to reduce a number of possible syringes used in the pump, or to double-check or aid in verifying that a correct syringe has been selected for use in the pump. Such a feature could provide greater infusion safety by minimizing a chance of delivering an incorrect amount of medication to the patient. In this regard, a particular syringe pump may only be able to measure an external diameter of a syringe based on, e.g., travel or displacement of a syringe barrel holder or clamp in the pump when the syringe is installed in the pump; and various types and sizes of syringes may have similar outer or external diameters but dissimilar internal diameters. For example, some 3 mL and 1 mL syringes have similar external diameters but significantly different internal diameters. In an embodiment of this feature of syringe internal diameter detection provided by a pump-actuatable clamp system as described by example or otherwise contemplated herein, the system could function analogously to the aforedescribed improved startup performance feature. In particular, the pump could use its occlusion pressure sensor to detect a first pressure. With the pump-actuatable clamp system occluding the tubing, the pump's motor could then be run to advance the pump's syringe plunger driver until the occlusion pressure sensor detects a second pressure that is of a selected higher pressure than the first pressure. A suitable microprocessor could be employed by or in the pump, to calculate the second pressure versus forward displacement of the pump's syringe plunger driver for a particular syringe to approximately determine the internal diameter of the syringe. In an embodiment, such calculation could take into account selected physical parameters such as distance of travel of the plunger driver relative to the sensed occlusion pressure. A forward displacement of the syringe plunger driver that does not result in an increase of pressure to the second pressure that was expected or predicted by the microprocessor relative to selected physical syringe characteristics (e.g., external diameter, length, etc.) could thereby result in a conclusion or alarm that an incorrect syringe may have been selected and installed in the pump.

Although described with particular reference to syringe pumps, it is to be appreciated and understood that the novel and inventive pump-actuatable clamp systems that have been described by example or are otherwise contemplated herein may also be used with any suitable infusion pumps (such as, for example, so-called ambulatory pumps, large volume pumps, peristaltic pumps, and elastomeric pumps, etc.) provided that suitable components and systems thereof satisfactorily function in cooperation with the pump-actuatable clamp systems according to subject matter hereof.

In some embodiments, a pump-actuatable clamp can provide or participate in additional functionality. For example, in some embodiments, a pump-actuatable clamp attached to an infusion line can be configured to facilitate electrical and/or optical connectivity between the infusion pump and the infusion line by either providing electrical and/or optical connectivity via the pump-actuatable clamp. U.S. Patent Application Publication No. 2013/0123579, “ILLUMINATED TUBING SET,” and U.S. Patent Application Publication No. 2013/0123743, “MEDICAL TUBING DETECTION AND MANAGEMENT” describe, in part, infusion lines that include optical elements that can provide illumination. The present disclosure contemplates infusion pumps that include power sources configured to provide power to such an optical element of an infusion line. A pump-actuatable clamp of an infusion line similar to clamp 200 could facilitate coupling of power from the power source to the optical element in any suitable manner. For example, the power source could provide power to the pump-actuatable clamp via a clamp receptacle.

Various embodiments of systems, devices, and methods have been described herein. These embodiments are given only by way of example and are not intended to limit the scope of subject matter hereof. It should be appreciated, moreover, that the various features of the embodiments that have been described may be combined in various ways to produce numerous additional embodiments. Moreover, while various materials, dimensions, shapes, configurations and locations, etc. have been described for use with disclosed embodiments, others besides those disclosed may be utilized commensurate with the scope of subject matter hereof.

Persons of ordinary skill in the relevant arts will recognize that subject matter hereof may comprise fewer features than illustrated in any individual embodiment described above. The embodiments described herein are not meant to be an exhaustive presentation of the ways in which the various features of subject matter hereof may be combined. Accordingly, the embodiments are not mutually exclusive combinations of features; rather, the subject matter hereof may comprise a combination of different individual features selected from different individual embodiments, as understood by persons of ordinary skill in the art.

Any incorporation by reference of documents above is limited such that no subject matter is incorporated that is contrary to the explicit disclosure herein. Any incorporation by reference of documents above is further limited such that no claims included in the documents are incorporated by reference herein. Any incorporation by reference of documents above is yet further limited such that any definitions provided in the documents are not incorporated by reference herein unless expressly included herein.

For purposes of interpreting the claims of subject matter hereof, it is expressly intended that the provisions of Section 112, sixth paragraph of 35 U.S.C. are not to be invoked unless the specific terms “means for” or “step for” are recited in a claim.

1. An infusion line clamp system, comprising:
a clamp configured to attach to an infusion line and reversibly occlude the infusion line, the clamp being biased to occlude the infusion line except when acted upon by an externally applied opening force; and
an infusion pump, including:
a control system;
a clamp receptacle configured to releasably secure the clamp to the infusion pump; and
a clamp actuation mechanism configured to selectively actuate the clamp under command of the control system by selective application of opening force to the clamp, when the clamp is secured to the pump.

2. The system of claim 1, configured such that when the clamp is removed from the clamp receptacle, any opening force that had been applied by the clamp actuation mechanism to the clamp is removed from the clamp and the clamp then responsively occludes, or remains occluding, the infusion line.

3. The system of claim 1, wherein the clamp actuation mechanism includes a solenoid or a motor.

4. The system of claim 1, wherein the infusion pump is a syringe pump.

5. The system of claim 1, wherein the control system is configured and programmed, upon occurrence of a predetermined safety condition, to remove opening force from the clamp via the clamp actuation mechanism, thereby occluding the infusion line.

6. The system of claim 5, wherein the infusion pump includes an accelerometer communicatively coupled to the control system, further wherein the predetermined safety condition is detected at least in part based upon acceleration information measured by the accelerometer.

7. An infusion pump, comprising:
a control system;
a clamp receptacle configured to releasably secure a clamp to the pump, wherein the clamp is configured to attach to an infusion line and reversibly occlude the infusion line, the clamp being biased to occlude the infusion line except when acted upon by an externally applied opening force;
a clamp actuation mechanism configured to selectively actuate the clamp under command of the control system by selective application of opening force, when the clamp is secured to the pump;
a syringe receptacle configured to receive a syringe, the syringe being coupleable to the infusion line; and
a pusher mechanism that can act to move a plunger of the syringe.

8. The system of claim 7, wherein the clamp actuation mechanism includes a solenoid or a motor.

9. The system of claim 7, wherein the control system is configured and programmed, upon occurrence of a predetermined safety condition, to remove opening force from the clamp via the clamp actuation mechanism, thereby occluding the infusion line.

10. The system of claim 7, wherein the infusion pump includes an accelerometer communicatively coupled to the control system, further wherein the predetermined safety condition is detected at least in part based upon acceleration information measured by the accelerometer.

11.-16. (canceled)

17. An infusion line clamp, comprising:
a first member and a second member separated by a space into which a portion of a compressible infusion line can be placed;
a biasing mechanism configured to urge the first and second members together such that the infusion line is compressively clamped between the first and second members with sufficient force to temporarily and reversibly occlude the infusion line;
mounting structures configured to interface with a clamp receptacle of an infusion pump, such that the infusion line clamp can be releasably secured to the infusion pump; and
at least one opening force receiving portion configured to receive opening force exerted by a clamp actuation mechanism of the infusion pump, such that when sufficient opening force is exerted on the at least one opening force receiving portion, the biasing mechanism is overcome and the first and second members are moved apart to open the infusion line to fluid flow.

18. (canceled)

19. An infusion line clamp system as claimed in claim 1, said infusion pump providing for a method of operation selected from a group as disclosed and described herein of:
preventing “crosstalk”;
improving startup performance;
running a pump motor in reverse to pull backwardly on a syringe plunger and thereby mitigate any unintended bolus of infusate that would otherwise be delivered from the syringe;
providing a test of the pump motor;
providing a test of a downstream occlusion sensor;
providing a test of motor health;
providing a test of motor rate error prevention;
determining a presence and amount of air in infusion line;
determining whether there may be a leak or a misconnection somewhere in the infusate's flow path;
estimating an internal diameter of a syringe; and
estimating a fluid volume capacity of a syringe.

20. An infusion pump as claimed in claim 7, the infusion pump including a pump-actuatable clamp system, the method of operation of said infusion pump selected from a group as disclosed and described herein of:
preventing “crosstalk”;
improving startup performance;
running a pump motor in reverse to pull backwardly on a syringe plunger and thereby mitigate any unintended bolus of infusate that would otherwise be delivered from the syringe;
providing a test of the pump motor;
providing a test of a downstream occlusion sensor;
providing a test of motor health;
providing a test of motor rate error prevention;
determining a presence and amount of air in infusion line;
determining whether there may be a leak or a misconnection somewhere in the infusate's flow path;
estimating an internal diameter of a syringe; and
estimating a fluid volume capacity of a syringe.

21. An infusion line clamp as claimed in claim 17, the infusion pump including a pump-actuatable clamp system for the infusion pump and a method of operation selected from a group as disclosed and described herein of:
preventing “crosstalk”;
improving startup performance;
running a pump motor in reverse to pull backwardly on a syringe plunger and thereby mitigate any unintended bolus of infusate that would otherwise be delivered from the syringe;
providing a test of the pump motor;
providing a test of a downstream occlusion sensor;
providing a test of motor health;
providing a test of motor rate error prevention;
determining a presence and amount of air in infusion line;
determining whether there may be a leak or a misconnection somewhere in the infusate's flow path;
estimating an internal diameter of a syringe; and
estimating a fluid volume capacity of a syringe.
